(19) United States
(12) Patent Application Publication
(10) Pub. No.: US 2021/0088346 A1
(43) Pub. Date: Mar. 25, 2021

(54) METHOD FOR DETERMINING A PREFERRED DESTINATION LOCATION FOR A VEHICLE

(71) Applicant: Siemens Healthcare GmbH, Erlangen (DE)

(72) Inventor: Stefan LAUTENSCHLAEGER, Nuernberg (DE)

(73) Assignee: Siemens Healthcare GmbH, Erlangen (DE)

(21) Appl. No.: 17/022,266

(22) Filed: Sep. 16, 2020

(30) Foreign Application Priority Data
Sep. 25, 2019 (EP) 19199605.7

(51) Int. Cl. G01C 21/34; G06T 7/00

(52) U.S. Cl. CPC G01C 21/3476; G06T 7/0012; G06T 2207/10081; G06T 2207/30101; G06T 2207/20081; G01C 21/3407

(57) ABSTRACT

A method is for determining a preferred destination location for a vehicle including a medical imaging device. In an embodiment, the method includes providing, via a first interface, an image data set relating to a patient, the image data set being created via the medical imaging device; evaluating the image data set provided via a computing unit; firstly determining a position of the vehicle via the computing unit; secondly determining, via the computing unit, a number of optional destination locations for treatment of the patient, at least based upon the position of the vehicle determined; thirdly determining, via the computing unit, the preferred destination location from the number of optional destination locations determined, at least based upon the image data set evaluated and the position of the vehicle determined; and outputting the preferred destination location determined via a second interface.

### PRIORITY STATEMENT

The present application hereby claims priority under 35 U.S.C. § 119 to European patent application number EP 19199605.7 filed Sep. 25, 2019, the entire contents of which are hereby incorporated herein by reference.

### FIELD

Embodiments of the invention generally relate to a method for determining a preferred destination location for a vehicle having a medical imaging device, a vehicle, a computer program product, a computer program and a computer-readable storage medium.

### BACKGROUND

For the purpose of treating a trauma patient or a stroke patient, a diagnosis is typically carried out by a doctor with the aid of medical image data sets. In order to allow a complete analysis of the medical situation of a patient, three-dimensional volume image data is often provided via a computer tomography device (CT device). This means that the imaging process involves data from a number of projection measurements recorded from different angular ranges, followed by a mathematical back-transformation in order to reconstruct the three-dimensional volume image data based upon the projection measurement data (e.g. via a reconstruction algorithm for filtered back projection). Based upon this three-dimensional volume image data, it is also possible to generate two-dimensional slice images, each representing a sectional image through the mapped volume.

In the case of a serious trauma or stroke, time is an important factor. The earlier the correct treatment starts, the better the prospects of successful treatment. Recent developments allow CT devices to be used more flexibly, e.g. in ambulances (so-called Mobile Stroke Units) or even in helicopters, thereby minimizing the time required for examination and diagnosis. In this case, a doctor (e.g. a neuroradiologist) is however not typically present in the ambulance to perform the diagnostic evaluation of the image data sets, and therefore the recorded image data sets must be transferred for example to a hospital for diagnosis.

### SUMMARY

The inventors have discovered, however, that this data transfer, usually via a mobile radio connection, usually represents a time-intensive factor which delays prompt assessment by a doctor. Depending on the assessment result, the steps that are subsequently necessary for the treatment of a patient may turn out to be different, however. Likewise, depending on the required treatment, different treatment locations will be preferable for the success of treatment or with regard to cost efficiency, e.g. according to equipment and availability.

For example, hospitals and/or stroke centers (or stroke units) can be classified into different levels of equipment or treatment according to their treatment options and structures that are available for the treatment of stroke patients. In the USA in particular, it is possible to distinguish between primary stroke centers (PSC) and comprehensive stroke centers (CSC) or thrombectomy capable stroke centers (TCSC). Similar hierarchies are also applied in other countries and regions. A stroke patient who has, for example, a vessel blockage of a large brain artery (large vessel occlusion: LVO) requires a different equipment level, in particular a CSC/TCSC level, than a stroke patient for whom this is not the case and for whom a PSC level is not only sufficient for subsequent treatment but is also significantly more cost-efficient in this case, or for whom even the suspicion of a stroke is not confirmed.

At least one embodiment of the invention provides an improved method for determining a preferred destination location for a vehicle, in particular an ambulance, having a medical imaging device, in order to allow optimal treatment of a patient whenever possible.

Advantageous and in some cases per se inventive embodiment variants and developments of the invention are set forth in the claims and in the following description.

Embodiments are described below, both in relation to the claimed method and in relation to an inventive apparatus in the form of the claimed vehicle. Features, advantages or alternative embodiment variants cited in this context apply equally to the other claimed subject matter and vice versa. In other words, the material claims (e.g. relating to an apparatus, i.e. the claimed vehicle) can also be developed by the features that are described or claimed in connection with a method. The corresponding functional features of the method are embodied by corresponding material modules in this case.

At least one embodiment of the invention relates to a method for determining a preferred destination location for a vehicle, in particular an ambulance, having a medical imaging device, wherein the method comprises the provision of an image data set relating to a patient and created via the medical imaging device via a first interface, and the evaluation of the provided image data set via a computing unit. Furthermore, the inventive method comprises a first determination via the computing unit of a position of the vehicle, a second determination via the computing unit of a number of optional destination locations for treatment of the patient at least based upon the determined position of the vehicle, and a third determination via the computing unit of the preferred destination location from the number of optional destination locations at least based upon the evaluated image data set and the determined position of the vehicle. This is followed by a step comprising the output of the selected preferred destination location via a second interface.

At least one embodiment of the invention further relates to a vehicle, in particular an ambulance, comprising

a medical imaging device, which is designed to create an image data set of a patient,

a first interface, which is designed to make the created image data set available for subsequent processing,

a computing unit, which is designed to
evaluate the created image data set,
determine a position of the vehicle,
determine a number of optional destination locations for treatment of the patient, at least based upon the determined position of the vehicle, and
select a preferred destination location from the number of optional destination locations, at least based upon the evaluated image data set and the determined position of the vehicle, and

a second interface, which is designed to output the selected preferred destination location.

At least one embodiment of the invention further relates to a computer program product comprising a computer program which can be loaded directly into a storage unit of a computing unit of a vehicle, in particular an ambulance, with program sections for executing all steps of an embodiment of the method for determining a preferred destination location for a vehicle when the program sections are executed by the computing unit.

In at least one embodiment, the computer program product is stored e.g. on a computer-readable medium or on a network or server, from where it can be loaded into the processor of a data processing unit, wherein the processor can be directly connected to the data processing unit or designed as part of the data processing unit. Control information of the computer program product can also be stored on an electronically readable data medium. The control information on the electronically readable data medium can be configured such that it performs a method according to an embodiment of the invention when the data medium is used in a processing unit. Examples of electronically readable data media include a DVD, magnetic tape or a USB stick on which is stored electronically readable control information, in particular software. When this control information is read from the data medium and stored in a processing unit, all of the inventive embodiment variants of the methods described above can be performed. An embodiment of the invention can therefore also relate to the computer-readable medium and/or to the electronically readable data medium.

At least one embodiment of the invention further relates to a computer program which can be loaded directly into a storage unit of a computing unit of a vehicle, in particular an ambulance, with program sections for executing all steps of a method for determining a preferred destination location for a vehicle when the program sections are executed by the computing unit.

At least one embodiment of the invention further relates to a computer-readable storage medium on which are stored program sections that can be read and executed by a computing unit in order to execute all steps of a method for determining a preferred destination location for a vehicle when the program sections are executed by the computing unit.

At least one embodiment of the invention is further directed to a method for determining a preferred destination location for a vehicle including a medical imaging device, comprising:

providing, via a first interface, an image data set relating to a patient, the image data set being created via the medical imaging device;

evaluating the image data set provided via a computing unit;

firstly determining a position of the vehicle via the computing unit;

secondly determining, via the computing unit, a number of optional destination locations for treatment of the patient, at least based upon the position of the vehicle determined;

thirdly determining, via the computing unit, the preferred destination location from the number of optional destination locations determined, at least based upon the image data set evaluated and the position of the vehicle determined; and

outputting the preferred destination location determined via a second interface.

At least one embodiment of the invention is further directed to a vehicle, comprising:

a medical imaging device, designed to create an image data set of a patient;

a first interface, designed to make the image data set created, available for subsequent processing;

at least one processor, designed to

evaluate the image data set created,

determine a position of the vehicle,

determine a number of optional destination locations for treatment of the patient, at least based upon the position of the vehicle determined,

select a preferred destination location from the number of optional destination locations, at least based upon the image data set evaluated and the position of the vehicle determined; and

a second interface, designed to output the preferred destination location selected.

At least one embodiment of the invention is further directed to a storage unit of a computing unit of a vehicle, storing a computer program including program sections for executing the method of an embodiment when the program sections are executed by the computing unit.

At least one embodiment of the invention is further directed to a non-transitory computer program product storing a computer program, directly loadable into a storage unit of a computing unit of a vehicle, including program sections for executing the method of an embodiment when the program sections are executed by the computing unit.

At least one embodiment of the invention is further directed to a non-transitory computer-readable storage medium storing program sections, readable and executable by a computing unit to execute the method of an embodiment when the program sections are executed by the computing unit.

### BRIEF DESCRIPTION OF THE DRAWINGS

The invention is explained below with reference to example embodiment variants and the appended figures. The illustrations in the figures are schematic, much simplified and not necessarily to scale.

FIG. 1 shows a schematic method sequence of a method for determining a preferred destination location for a vehicle,

FIG. 2 shows a schematic method sequence of a further variant of a method for determining a preferred destination location for a vehicle,

FIG. 3 schematically shows an illustration of a number of optional destination locations,

FIG. 4 shows a schematic illustration of a vehicle comprising a medical imaging device.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for determining a preferred destination location for a vehicle, in particular an ambulance, having a medical imaging device, wherein the method comprises the provision of an image data set relating to a patient and created via the medical imaging device via a first interface, and the evaluation of the provided image data set via a computing unit. Furthermore, the inventive method comprises a first determination via the computing unit of a position of the vehicle, a second determination via the computing unit of a number of optional destination locations for treatment of the patient at least based upon the determined position of the vehicle, and a third determination via the computing unit of the preferred destination location from the number of optional destination locations at least based upon the evaluated image data set and the determined position of the vehicle. This is followed by a step comprising the output of the selected preferred destination location via a second interface.

At least one embodiment of the invention proceeds from the notion that in the context of a vehicle, in particular an ambulance, having a medical imaging device, it is possible at least based upon a first evaluation, preferably performed automatically via the computing unit, of the provided image data set of the patient, to determine an optimal treatment location for the patient, i.e. a preferred destination location for the vehicle, so that a compatible optimal treatment of the patient at the treatment location can be ensured. Optimal in this case can take into consideration the treatment success in particular, but also parameters such as the cost efficiency. In this case, it is intended preferably to avoid the necessity of transferring the image data set to a central unit, i.e. a hospital for example, for initial evaluation and diagnosis based upon the image data set, so that no time delay is involved and the patient reaches the appropriate treatment location as promptly as possible.

The medical imaging device can comprise e.g. an imaging device based on X-radiation, e.g. a CT device, a C-arm X-ray device or an angiography X-ray device. Other medical imaging devices designed to create a two-dimensional or even three-dimensional image data set of the patient are also conceivable, however.

The provision of the image data set can comprise e.g. capture and/or readout from a computer-readable data store and/or receipt from a storage unit via the first interface. In addition to this, the inventive method can also comprise the creation of the image data set via the medical imaging device, which image data set can then be provided for the further steps of the method via the first interface.

The image data set can be in particular a three-dimensional image data set (3D image data set) or a two-dimensional image data set (2D image data set). A 2D image data set allows a two-dimensional, in particular spatially two-dimensional, representation of an examination region of the patient. A 3D image data set allows in particular a three-dimensional, in particular spatially three-dimensional, representation of an examination region of the patient. A three-dimensional image data set can also be represented as a number of slice image data sets. A slice image data set comprises in each case a slice of the 3D image data set at a position along a marked axis. Therefore a slice image data set in each case allows a two-dimensional, in particular spatially two-dimensional, representation of the respective slice of the 3D image data set. A 3D image data set advantageously comprises a plurality of voxels, in particular image points. In this case, each voxel can preferably have a value, in particular an image value, e.g. a grayscale value and/or an RGB color value and/or an intensity value. Similarly, a two-dimensional image data set can comprise a plurality of pixels, in particular image points. In this case, each pixel can preferably have a respective value, in particular an image value, e.g. a grayscale value and/or an RGB color value and/or an intensity value.

The image data set can be an angiography image data set, an image data set without contrast medium support, or a perfusion image data set, for example.

The patient can be a human patient and/or an animal patient. An examination region of the patient comprised, i.e. represented, in the image data set, can include an anatomical and/or spatial region of the patient, comprising a predetermined tissue region and/or a spatial region that is required for a diagnosis. The examination region in this case can comprise a body region, e.g. the head or the thorax, of the patient. The examination region can also comprise the entire body of the patient if necessary. The patient is e.g. a stroke patient, meaning a patient who has suffered a stroke, i.e. in particular an ischemic infarction, but also a patient having at least a suspected stroke. The examination region can then comprise at least a part-region of the head of the stroke patient.

The evaluation is based in particular on an analysis of the first image data set via the computing unit. The analysis can be based on the image values or structures mapped thereby, e.g. edges, contiguous groups of image values, variations between adjacent image values, or changes of image values over time, etc. The evaluation or the analysis can comprise a threshold value method, a segmentation method or similar. In this case, the evaluation or the analysis can include a comparison with expected image values or expected structures, from which an evaluation or an analysis result can be derived. Expectations may be based e.g. on the image data set itself, possibly on a comparison of different regions of the image data set, on an anatomical atlas or model, or even on a plurality of similar previously existing image data sets of the patient or other patients. For example, a trained function can be applied for the purpose of the evaluation or at least partial aspects of the evaluation.

The evaluation of the provided image data set via the computing unit can comprise classification of the image data set into at least one evaluation group, at least for the purpose of grouping. This means that the provided image data set can check the image data set to establish whether the image data set can be assigned to the at least one evaluation group. The grouping can be based on the analysis of the first image data set in this case. The evaluation of the provided image data set via the computing unit can also comprise grading the image data set into a plurality of different categories, i.e. evaluation groups. The evaluated image data set or an evaluation group can be associated e.g. with a possible diagnosis, a necessary treatment step or a piece of medical equipment that is required for treatment of the patient. In addition to this, there are also other associations. A diagnosis may be the presence of a vessel blockage or the severity or extent of a vessel blockage, the presence of a cerebral hemorrhage or other. For example, the evaluation comprises checking the image data set for the presence of a vessel blockage, in particular for the presence of an LVO. The evaluation can also comprise the evaluation of checking the image data set for the presence of a cerebral hemorrhage or other impairment. The evaluation can also comprise situating the vessel blockage or cerebral hemorrhage in the first image data set, or determining the type of blood vessel affected by a vessel blockage. In particular, an evaluated image data set or an evaluation group can be associated with at least one criterion that must be satisfied for optimal treatment of the patient at a treatment location.

The evaluation can preferably be performed via the computing unit automatically and without further input from an operator or at least semiautomatic ally based upon the provided image data set. For example, provision can be made for an operator to start or stop the inventive method, confirm a grouping, or trigger further steps based upon the evaluation.

In the first determination step, the position of the vehicle can be determined via a position determining unit or requested from the position determining unit, e.g. a satellite-based position determining system such as GPS or the European global satellite navigation system Galileo. The vehicle can then be located on a map, e.g. in combination with map data. For this purpose, the vehicle can have a position determining unit, e.g. in the form of a GPS receiver, which allows the position of the vehicle to be determined. The position determining unit can allow the position of the vehicle to be requested via the computing unit for the first determination step. For example, the vehicle comprises a navigation system comprising a position determining unit, wherein the determination of the position of the vehicle via the computing unit comprises requesting the position from the navigation system.

The second determination comprises determining a number of optional destination locations. An optional destination location for the vehicle is in particular one of the possible treatment locations for the patient which the vehicle can reach, i.e. head for. A treatment location can generally be a hospital or other patient care center which is equipped for the treatment of patients, in particular emergency patients. If the method is restricted to stroke patients, the treatment locations may comprise only stroke centers, for example. These can correspond to hospitals that are equipped for the treatment of stroke patients in particular. It may be possible, for example, to identify a respective treatment location and hence a respective destination location by virtue of its location information. The location information may be present, for example, in the form of location coordinates, such as GPS coordinates, or also the address thereof. The number of optional destination locations that are determined in the second determination step can represent a selection of treatment locations. The optional destination locations are included in the treatment locations in particular, and represent at least a subset of the treatment locations.

The determined number of optional destination locations is determined at least based upon the determined position of the vehicle. The number of optional destination locations includes e.g. those treatment locations which lie within a determined surrounding area, within a determined travel distance or within a determined travel time starting from the position of the vehicle. Methods for calculating a destination within a surrounding area, a route or travel distance to a determined destination or a prediction of a travel time required to reach the destination are already known in the field of navigation systems and are therefore not explained further here.

The number of optional destination locations can also be determined with reference to further criteria, e.g. an availability of the medical equipment at a treatment location or an equipment level. The number of optional destination locations comprises e.g. a number of treatment locations currently available for treatment of the patient or a number of treatment locations having a determined equipment level. The number of optional destination locations comprises e.g. a number of CSCs/TCSCs and/or a number of PSCs which are identified within a determined surrounding area around the position of the vehicle.

For the purpose of determining the number of optional destination locations, a local database of treatment locations in the form of e.g. a table or list with the respective location information can be available locally on a storage unit of the computing unit or vehicle, the database being available for retrieval and processing by the computing unit for the second determination step. Each treatment location can also be associated with further information, e.g. an equipment level or similar, which is retrievably held in the database. Alternatively or additionally, the second determination of the number of optional destination locations can also comprise a request to a central unit. The central unit may be situated at a hospital or other site, e.g. a computer center. The central unit can likewise hold a database of treatment locations, which database can be queried e.g. via a radio network, in particular a mobile radio network. The central unit can hold additional retrievable information about treatment locations. In particular, the central unit can also provide information associated with the treatment locations, wherein the information held on the central unit can also be updated relative to time in particular. For example, the current availability of the medical equipment at a treatment location can be requested via the central unit. For this purpose, the vehicle or the computing unit can provide in particular a communication unit, which is designed to implement a request to a central unit, in particular via a radio network.

The preferred destination location is determined or selected from the number of optional destination locations via the computing unit based upon the evaluated image data set.

The preferred destination location can be determined with reference to at least one criterion. The evaluated image data set can be associated with at least one criterion that preferably must be satisfied by an optional destination location for treating the patient. The at least one criterion can be correlated with the determined optional destination locations. The preferred destination location can be determined based upon the correlation. Depending on the evaluation, a different optional destination location can be determined as the preferred destination location. If the evaluation of the image data set is associated e.g. with a possible diagnosis, a necessary treatment step or medical equipment that is required for treatment of the patient, a required piece of medical equipment can be directly ascertained or derived therefrom as a criterion. This criterion can then be correlated with the determined optional destination locations, based upon which the preferred destination location can be determined. A further criterion may be the destination location most rapidly accessible by the vehicle. The accessibility can be calculated with reference to a travel distance or a predicted travel time, which can be determined with reference to the position of the vehicle and the optional destination locations. A further criterion can be e.g. the availability of the medical equipment. Further criteria for a correlation are also conceivable.

The output of the selected preferred destination location can comprise the output of the destination location to an output unit via the second interface. The output unit can be designed to output or noticeably provide the destination location to a driver of the vehicle by visual or acoustic device(s). The output unit can comprise e.g. a display or a loudspeaker. The output of the selected preferred destination location can comprise the output of the destination location via the second interface to a navigation system which is included in the vehicle or the computing unit.

The preferred destination location can be output e.g. in the form of location information, i.e. an address, or in the form of location coordinates or other format.

The inventors have recognized that the method according to at least one embodiment of the invention can advantageously ensure particularly time-efficient and optimized treatment of a patient, in that an evaluation is performed based upon the first image data set via a computing unit which is held locally in the vehicle, and a compatible optimal treatment location is determined as a preferred destination location for the vehicle. A time delay can advantageously be avoided by transferring the image data set for telemedical evaluation. It is advantageously possible in each case to select a treatment location that is optimal for treating the patient in respect of accessibility, equipment availability and cost efficiency.

In a preferred variant embodiment of the method, at least one parameter from the following list is determined in the second determination step:

medical equipment at a respective optional destination location for treating the patient,

availability of the medical equipment at a respective optional destination location for treating the patient,

travel route to a respective optional destination location,

travel time to a respective optional destination location.

The at least one determined parameter can be fed into the second determination step of the optional destination locations and/or into the third determination step of the preferred destination location. This can mean that the number of optional destination locations can be limited based upon at least one of these parameters. The at least one parameter is preferably fed in as a criterion for determining the preferred destination location from the number of optional destination locations.

The medical equipment can be described e.g. by grading the treatment location or the optional destination location based upon equipment levels that have been determined. This information can be made available to the computing unit in a retrievable manner, together with the location information of the treatment locations. For example, at least CSCs/TCSCs can be distinguished from PSCs. Other distinctions or gradings can also be used. The medical equipment at the treatment locations can also be specified in greater detail, and describe the different treatment options in the form of the medical devices that are available and/or in the form of the medical staff who are present and trained for a given treatment at the treatment location, for example, as retrievable information in each case.

The availability of the medical equipment can include a loading factor of the treatment possibilities, i.e. the medical devices and/or the medical staff at a respective treatment location. In particular, the availability can be requested as information that is updated relative to time. For example, it is possible to determine only available treatment locations as optional destination locations in the second determination step. For example, it is possible to determine only an available optional destination location as a preferred destination location in the third determination step.

In particular, the at least one parameter can be determined automatically via the computing unit. The at least one parameter can be requested from a local database which holds a treatment location associated with the at least one parameter in each case. A request to a central database at a central unit can also be included.

By determining a travel route to a respective treatment location or to a respective optional destination location, conclusions about a travel distance can be made. A determined travel route can moreover also allow conclusions about a predicted travel time. A predicted travel time can also include a current or forecast traffic volume on the travel route. For example, it is possible to determine only optional destination locations within a determined travel distance or travel time. For example, the preferred destination location can be determined based upon the shortest travel distance or the shortest travel time. The travel route or the predicted travel time can be determined via a navigation system contained in the vehicle or in the computing unit, or can be requested from such a navigation system via the computing unit for this purpose. Methods for determining or predicting a travel route or a travel time based thereon are widely used in the field of navigation systems and therefore need not be explained in detail here.

The parameters can be combined for the purpose of determining the preferred destination location, in particular in a manner which is compatible with the evaluated image data set. For example, depending on the evaluation of the image data set, it is possible to select as the preferred destination that available optional destination location which has a required equipment level and can be reached in the shortest possible time by the vehicle.

The determination of at least one of the above parameters advantageously allows the computing unit to determine the optional destination locations and in particular select the preferred destination location in a particularly advantageous manner, such that an optimal and time-efficient treatment can be achieved.

In a further advantageous method variant embodiment, the evaluation step comprises checking the provided image data set for the presence of a vessel blockage of a blood vessel of the patient.

In particular, the evaluation step can comprise checking the provided image data set for the presence of a vessel blockage of a blood vessel in the brain of the patient. The presence of a vessel blockage, in particular in the brain of the patient, is associated in particular with the need for particularly rapid and optimal treatment of a patient, and therefore the advantages of the inventive method can contribute in a particularly advantageous manner to a successful treatment.

The checking in the evaluation step can include establishing the presence of a vessel blockage in the examination region. This means that an evaluated image data set can be evaluated solely in respect of whether a vessel blockage is present in the examination region. The presence of a vessel blockage can for example be associated with or correspond to an evaluation group into which the image data set can be grouped or classified in the evaluation step.

The checking can also include situating the vessel blockage. This means that an evaluated image data set can be evaluated in respect of whether a vessel blockage is present and where a vessel blockage is present in the first image data set or in the examination region. The location of the vessel blockage can allow an indication with regard to the consequences of the vessel blockage or to necessary treatment steps, and therefore give important indications with regard to the evaluation or grouping into a corresponding evaluation group and therefore also an optimal treatment. The location of a vessel blockage can be associated e.g. with an evaluation group into which the image data set can be grouped or classified in the evaluation step.

In this case, the checking can also include determining the blood vessel or the type of blood vessel in which the vessel blockage is present. For example, it is possible to distinguish between different blood vessels for the evaluation. The different blood vessels or blood vessel types can also have roles of differing importance in the blood supply of the brain of the patient. The blood vessel type in which a vessel blockage is present may be associated e.g. with an evaluation group into which the image data set can be grouped or classified in the evaluation step.

Different locations or blood vessel types may be associated with different demands and requirements with regard to the treatment of a patient, and therefore feeding such information into the evaluation and then determining a preferred destination location on this basis can advantageously result in optimization of the treatment process.

In an advantageous method variant embodiment, if a vessel blockage is present, provision is made for determining a location of the vessel blockage or a type of blood vessel affected by the vessel blockage. For example, an LVO (i.e. a vessel blockage of a large brain artery) may be determined. For example, an LVO may be associated with an evaluation group into which the image data set can be grouped or classified in the evaluation step.

Depending on the evaluation, different equipment levels (i.e. different destination locations) may be required for the purpose of treating the patient. For example, a CSC/TCSC level of stroke center may be required in order to treat a patient in the case of an LVO in particular, whereas a PCS may be sufficient in the case of a vessel blockage of a small secondary blood vessel.

In this case, in an embodiment of the method, a machine learning method or trained function can be applied in the evaluation step.

Application of the trained function to the image data set provided can allow the image data set to be evaluated. The trained function can also be restricted to a partial aspect of the evaluation, e.g. the checking, situating and/or grading or grouping.

In this case, the trained function can advantageously be trained by a machine learning method. In particular, the trained function can be a neural network, in particular convolutional neural network (CNN) or a network comprising a convolutional layer.

A trained function maps input data onto output data. In this context, the output data can also depend in particular on one or more parameters of the trained function. The one or more parameters of the trained function can be determined and/or adjusted by way of training. The determination and/or adjustment of the one or more parameters of the trained function can be based in particular on a pair of training input data and associated training output data, wherein the trained function is applied to the training input data for the purpose of creating output data. In particular, the evaluation can be based on a comparison of the created output data with the training output data. A trainable function, i.e. a function with one or more parameters that have not yet been adjusted, is generally also referred to as a trained function.

Other terms for trained function include trained mapping rule, mapping rule with trained parameters, function with trained parameters, algorithm based on artificial intelligence, and machine learning algorithm. An example of a trained function is an artificial neural network, wherein the arc weights of the artificial neural network correspond to the parameters of the trained function. In particular, a trained function can also be a deep artificial neural network, also referred to as a deep neural network. A further example of a trained function is a ‘support vector machine’, and in particular other machine learning algorithms can also be used as a trained function. The trained function can be trained by way of back propagation, for example. Output data can first be created by applying the trained function to training input data. A variation between the created output data and the training output data can then be calculated by applying an error function to the created output data and the training output data. It is further possible iteratively to adjust a parameter, in particular a weighting, of the trained function, in particular the neural network, based upon a gradient of the error function in relation to the at least one parameter of the trained function. By this, the variation between the training mapping data and the training output data can advantageously be minimized during the training of the trained function.

The input data can comprise a multiplicity of image data sets created by the medical device. The training output data can comprise corresponding evaluated image data sets.

As a result of applying an artificial intelligence system, i.e. a trained function, all of the relevant influencing variables for the evaluation can be taken into consideration, including those for which a user is not able to assess a relevance to the evaluation. In particular, an evaluation can be achieved automatically in a particularly reliable and time-efficient manner by way of a trained function after the training phase. A telemedical evaluation and accompanying time delay can advantageously be avoided.

According to an embodiment of the inventive method, the created image data set can also be a CT angiography image data set.

A CT angiography image data set most advantageously allows an evaluation in respect of a vessel blockage of a blood vessel of a patient, in particular a stroke patient.

In an embodiment variant of the inventive method, the first determination step and the second determination step are executed repeatedly, such that an updated number of optional destination locations is determined if the position of the vehicle is updated.

If the position of the vehicle is updated, i.e. if the vehicle has moved, it is advantageously also possible to determine an optimal treatment location, i.e. an optimal destination location. For example, a current availability or a current predicted travel time can also be continuously fed into the third determination of the preferred destination location.

Furthermore, provision can be made for the first determination step and/or the second determination step to be performed during the evaluation step.

During can mean that the steps are performed, started or ended largely simultaneously or at least in temporal proximity. At the same time as the evaluation step, or at least temporally proximate to the completion thereof, i.e. while an evaluation is taking place, the number of optional destination locations can advantageously be determined and made available for the third determination step of the preferred destination location. A time-efficient process can advantageously be ensured.

In a further variant embodiment of the inventive method, navigation information for reaching the preferred destination location is calculated based upon the determined preferred destination location and output via an output unit.

For example, the computing unit or the vehicle comprises a navigation system in order to calculate navigation information for the driver of the vehicle for the purpose of reaching a navigation destination. In this way, the preferred destination location can automatically be programmed into the navigation system as the selected navigation destination via the interface. The driver can then be guided to the preferred destination location based upon navigation information provided by the navigation system. The navigation information can include visual and/or acoustic information. For example, the navigation information comprises map data on which the destination location and/or a route to the destination location starting from the current position of the vehicle is shown, or voice instructions. Accordingly, the output unit can therefore comprise a display for presentation of the navigation information to the driver or a loudspeaker for the acoustic output of the navigation information to the driver.

At least one embodiment of the invention further relates to a vehicle, in particular an ambulance, comprising

a medical imaging device, which is designed to create an image data set of a patient,

a first interface, which is designed to make the created image data set available for subsequent processing,

a computing unit, which is designed to
evaluate the created image data set,
determine a position of the vehicle,
determine a number of optional destination locations for treatment of the patient, at least based upon the determined position of the vehicle, and
select a preferred destination location from the number of optional destination locations, at least based upon the evaluated image data set and the determined position of the vehicle, and

a second interface, which is designed to output the selected preferred destination location.

The vehicle can be a so-called Mobile Stroke Unit, for example. A vehicle according to the invention can be designed in particular to execute the inventive method and aspects thereof as described above. The inventive vehicle can be designed to execute the method and aspects thereof, in that the medical imaging device, the computing unit and the interfaces are designed to execute the corresponding method steps.

The different configurations, features and advantages of the method and its embodiment variants can likewise be transferred directly to the inventive vehicle in this case.

In a preferred apparatus variant embodiment, the medical imaging device is a CT device in particular. In this case, the CT device can be designed in particular to create CT angiography image data sets and perfusion CT image data sets in addition to native CT image data sets.

At least one embodiment of the invention further relates to a computer program product comprising a computer program which can be loaded directly into a storage unit of a computing unit of a vehicle, in particular an ambulance, with program sections for executing all steps of an embodiment of the method for determining a preferred destination location for a vehicle when the program sections are executed by the computing unit.

The computer program product in this case can be software comprising source code which must then be compiled and linked or simply interpreted, or executable software code which merely has to be loaded into the computing unit for execution. By virtue of the computer program product, the method for controlling a medical imaging device can be executed in a manner which is quick, uniformly repeatable and robust. The computer program product is configured such that it can execute an embodiment of the inventive method steps via the computing unit. The computing unit in this case must meet the requirements for e.g. a corresponding main memory, a corresponding graphics card or a corresponding logic unit, so that the respective method steps can be executed efficiently.

The computer program product is stored e.g. on a computer-readable medium or on a network or server, from where it can be loaded into the processor of a data processing unit, wherein the processor can be directly connected to the data processing unit or designed as part of the data processing unit. Control information of the computer program product can also be stored on an electronically readable data medium. The control information on the electronically readable data medium can be configured such that it performs a method according to an embodiment of the invention when the data medium is used in a processing unit. Examples of electronically readable data media include a DVD, magnetic tape or a USB stick on which is stored electronically readable control information, in particular software. When this control information is read from the data medium and stored in a processing unit, all of the inventive embodiment variants of the methods described above can be performed. An embodiment of the invention can therefore also relate to the computer-readable medium and/or to the electronically readable data medium.

A largely software-based realization has the advantage that apparatus already used to control a medical imaging device can be upgraded easily via a software update in order to work in the inventive manner. In addition to the computer program, such a computer program product can optionally comprise additional parts such as e.g. documentation and/or additional components, and hardware components such as e.g. hardware keys (dongles etc.) for using the software.

At least one embodiment of the invention further relates to a computer program which can be loaded directly into a storage unit of a computing unit of a vehicle, in particular an ambulance, with program sections for executing all steps of a method for determining a preferred destination location for a vehicle when the program sections are executed by the computing unit.

At least one embodiment of the invention further relates to a computer-readable storage medium on which are stored program sections that can be read and executed by a computing unit in order to execute all steps of a method for determining a preferred destination location for a vehicle when the program sections are executed by the computing unit.

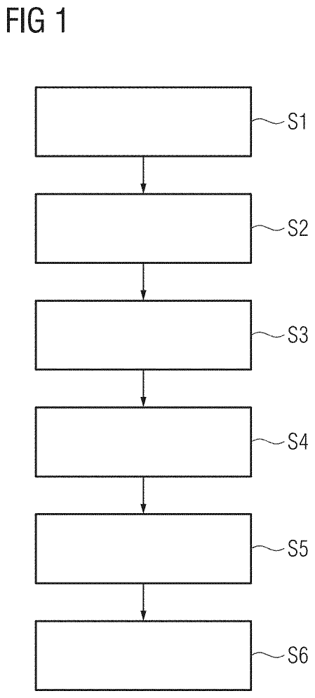

FIG. 1 shows a schematic method sequence of a method for determining a preferred destination location for a vehicle 100, in particular an ambulance, having a medical imaging device 101. For example, the medical imaging device is a CT device which is designed to create a three-dimensional image data set.

The method comprises the step of providing S1 an image data set relating to a patient 103, which image data set was created by the medical imaging device 101, via a first interface 105. The image data set can be an angiography image data set, an image data set without contrast medium support, or a perfusion image data set, for example.

Following thereupon, the provided image data set is evaluated via a computing unit 107 in an evaluation step S2. The evaluation in this case is based on an analysis of the first image data set via the computing unit 107. The analysis can be based on the image values or structures mapped thereby, e.g. edges, contiguous groups of image values, variations between adjacent image values or changes of image values over time, etc. The evaluation of the provided image data set via the computing unit 107 can include grouping the image data set at least into at least one evaluation group. This means that the provided image data set can check the image data set to establish whether the image data set can be assigned to the at least one evaluation group. In particular, an evaluated image data set or an evaluation group can be associated with at least one criterion that must be satisfied for optimal treatment of the patient 103 at a treatment location. A criterion can be e.g. the medical equipment at the treatment location, a determined equipment level at the treatment location or an availability of the treatment location. Other criteria can also be applied.

In this case, the step of evaluating S2 the image data set can include checking the image data set for the presence of a vessel blockage of a blood vessel of the patient 103, in particular in the brain of the patient 103. If a vessel blockage is present, a location of the vessel blockage or a type of the blood vessel affected by the vessel blockage can preferably also be determined. For example, the evaluation includes checking the image data set for the presence of a vessel blockage, in particular for the presence of an LVO.

It is particularly advantageous if the provided image data set is a CT angiography image data set. An evaluation of the image data set in respect of a vessel blockage can be achieved particularly advantageously via a CT angiography image data set.

In an advantageous variant, the evaluation step S2 comprises the application of a trained function in particular. As a result of applying an artificial intelligence system, i.e. a trained function, all of the relevant influencing variables for the evaluation can be taken into consideration, including those for which a user is not able to assess a relevance to the evaluation. In particular, an evaluation can be achieved automatically in a particularly reliable and time-efficient manner by way of a trained function after the training phase.

In a further, first determination step S3, a position 4 of the vehicle 100 is determined. In the first determination step, the position 4 of the vehicle 100 can be calculated via a position determining unit 113 or requested from the position determining unit 113, e.g. a satellite-based position determining system such as e.g. GPS or the European global satellite navigation system Galileo. The position determining unit 113 can then be implemented in the form of a GPS receiver, for example.

In a further, second determination step S4, a number of optional destination locations 1,2,3 for treatment of the patient 103 are determined at least based upon the position of the vehicle as determined via the computing unit 107. The number of optional destination locations 1,2,3 can be a selection of treatment locations, i.e. hospitals, stroke centers, or similar. A respective treatment location or destination location can be specified using the location coordinates or address thereof. The determined number of optional destination locations 1,2,3 is determined at least based upon the determined position of the vehicle. For example, the number of optional destination locations 1,2,3 comprises those treatment locations which lie within a determined surrounding area, within a determined travel distance or within a determined travel time starting from the position 4 of the vehicle 100. The number of optional destination locations 1,2,3 can also be limited based upon further criteria, e.g. medical equipment or availability of the medical equipment at a treatment location.

In a third determination step S5, the preferred destination location is then determined from the number of optional destination locations 1,2,3 via the computing unit 107 at least based upon the evaluated image data set and the determined position of the vehicle 100. The preferred destination location can be determined based upon at least one criterion, wherein the criterion can be derived in particular from the evaluation, i.e. the evaluated image data set. The evaluated image data set can be associated with at least one criterion which must preferably be satisfied by an optional destination location for treating the patient. The at least one criterion can be correlated with the determined optional destination locations 1,2,3. The preferred destination location can be determined based upon the correlation.

In an advantageous variant, at least one parameter from the following list is also determined in the second determination step S4:

medical equipment of a respective optional destination location 1,2,3 for treating the patient 103,

availability of the medical equipment of a respective optional destination location 1,2,3 for treating the patient 103,

travel route 5 to a respective optional destination location 1,2,3,

travel time to a respective optional destination location 1,2,3.

In this case, the at least one parameter can be fed into the second determination step S4 itself or also into the third determination step S5 of the preferred destination location, based on the number of optional destination locations 1,2,3. This can mean that the number of optional destination locations 1,2,3 can be limited based upon at least one of these parameters. The at least one parameter is preferably fed in as a criterion for determining S5 the preferred destination location from the number of optional destination locations 1,2,3.

For example, the optional destination locations 1,2,3 determined in the second determination step include available treatment locations with both CSC/TCSC level and PSC level. Depending on the evaluated image data set, either a CSC/TCSC or a PSC can be determined as a preferred destination location in the third determination step S5. For example, the CSC/TCSC or the PSC having the quickest access measured in a travel distance or in a predicted travel time is determined as a preferred destination location. For example, the presence of an LVO is detected during the evaluation of the image data set. The presence of an LVO can be associated with the need for a CSC/TCSC equipment level, whereupon the closest CSC/TCSC starting from the position of the vehicle is determined as a preferred destination location. Other scenarios which vary from this are also conceivable.

In a further step for the output S6, the selected preferred destination location is output via a second interface 109. The output of the selected preferred destination location can comprise the output of the preferred destination location to an output unit 119 via the second interface 109. The output unit 119 can be designed to output or noticeably provide the preferred destination location to a driver of the vehicle 100 by visual and/or acoustic device(s). The output unit 119 can comprise e.g. a display or a loudspeaker. The output of the selected preferred destination location can also comprise the output of the destination location via the second interface to a navigation system 115, this being included in the vehicle 100 or the computing unit and being designed to calculate navigation information for the purpose of reaching the preferred destination location and to make the navigation information visually and/or acoustically available to the driver.

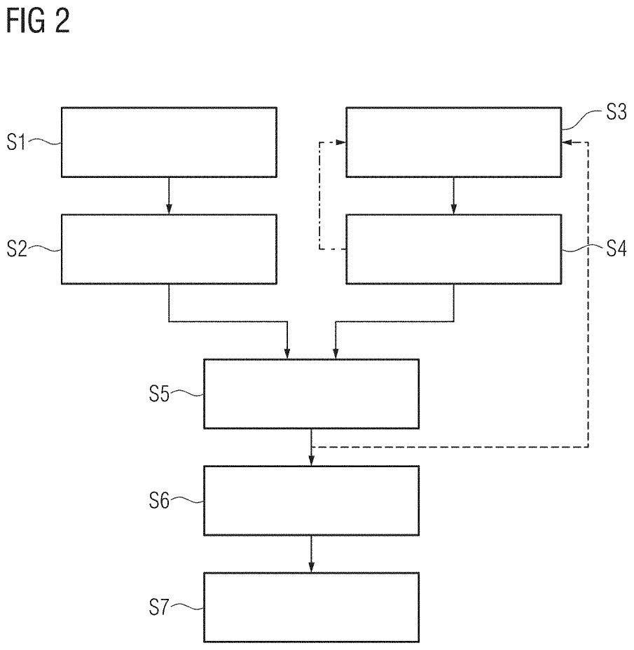

FIG. 2 shows a further variant of a method for determining a preferred destination location for a vehicle 100, in particular an ambulance, having a medical imaging device 101. In this example, the first determination step S3 and the second determination step S4 can preferably take place repeatedly, such that an updated number of optional destination locations 1,2,3 can be determined if a position 4 of the vehicle 100 is updated.

Furthermore, the first determination step S3 and the second determination step S4 take place at least to some extent at essentially the same time as the evaluation step S2, such that upon completion of the evaluation step S2 or at least temporally proximate thereto, a number of optional destination locations 1,2,3 can preferably already be provided for the third determination S5. Moreover, the first determination step S3 and/or the second determination step S4 can likewise already take place or be started at essentially the same time as or during a step of the creation of the first image data set S0.

It is also conceivable as indicated here by the broken-line arrow that a first determination S3 and second determination S4 are repeated even after a third determination step S5, such that in the event of changes to at least one parameter on which the third determination S5 of the preferred destination location is based, a current and possibly optimized preferred destination location can be output. Such a scenario may be, for example, a traffic jam which occurs unexpectedly due to an accident and potentially results in a delay, or a temporary change in the availability of the medical equipment at the destination location.

Furthermore, the example method shown here also has the step S7 of calculating navigation information for the purpose of reaching the preferred destination location, the navigation information being output e.g. in visual and/or acoustic form via an output unit 119, so that a driver of the vehicle 100 can be guided to the preferred destination location by way of the navigation information.

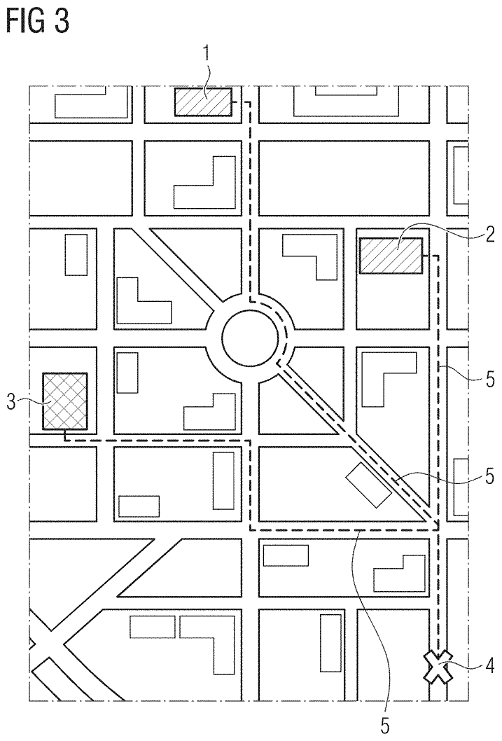

For illustrative purposes, FIG. 3 schematically shows a map representation on which a determined position 4 of the vehicle 100 is marked. Also highlighted by hatching are three example optional destination locations 1,2,3. The optional destination locations 1,2,3 respectively mark optional destination locations 1,2,3 for treating the patient, which have been determined at least based upon the position 4 of the vehicle.

The different hatching of the optional destination locations 1 and 2 compared with the optional destination location 3 is intended to illustrate in this case that the optional destination locations 1 and 2 have different medical equipment for example, e.g. a different equipment level, than the optional destination location 3. For example, the optional destination locations 1 and 2 are CSCs/TCSCs and the optional destination location 3 is a PSC. Similarly, the hatching can also symbolize different availabilities of the medical equipment or other parameters likewise. For example, the optional destination location 3 is currently not available to treat a patient due to the loading factor of the medical equipment at the treatment location.

Also illustrated are travel routes 5 to a respective optional destination location 1,2,3 of the number of optional destination locations 1,2,3. Based upon the travel route, it is optionally possible to determine a travel distance or even a predicted travel time, which can also include the current or predicted traffic volume on the travel route.

Depending on the evaluation of the image data set, the preferred destination location can then be selected from the number of optional destination locations 1,2,3. If the patient is a stroke patient and the evaluation of the image data set includes e.g. an LVO diagnosis, it is possible based on the current position 4 of the vehicle 100 to select and output as the preferred destination location that CSC/TCSC which can be reached most quickly and is available to provide treatment, e.g. destination location 2 or possibly also destination location 3 in the case outlined above. If the evaluation only includes a diagnosis which requires a lower equipment level, e.g. the PSC as destination location 3 in the case outlined above can be determined as the preferred destination location.

Based upon the determined preferred destination location, it is then possible to calculate navigation information for reaching the preferred destination location and to output the navigation information via an output unit. The navigation information can be provided to a driver as map data by way of a presentation similar to that shown schematically here in FIG. 3. For example, the computing unit or the vehicle 100 comprises a navigation system 115, wherein the preferred destination location is automatically programmed into the navigation system 115 as the selected navigation destination via the interface, and the navigation system 115 calculates the navigation information on this basis. In this case, the navigation information can be visual information, e.g. a map as illustrated here, which shows the preferred destination location and/or a route to the preferred destination location starting from the current position of the vehicle and is output via an output unit in the form of a display. The driver can then be guided to the preferred destination location with reference to navigation information provided by the navigation system 115.

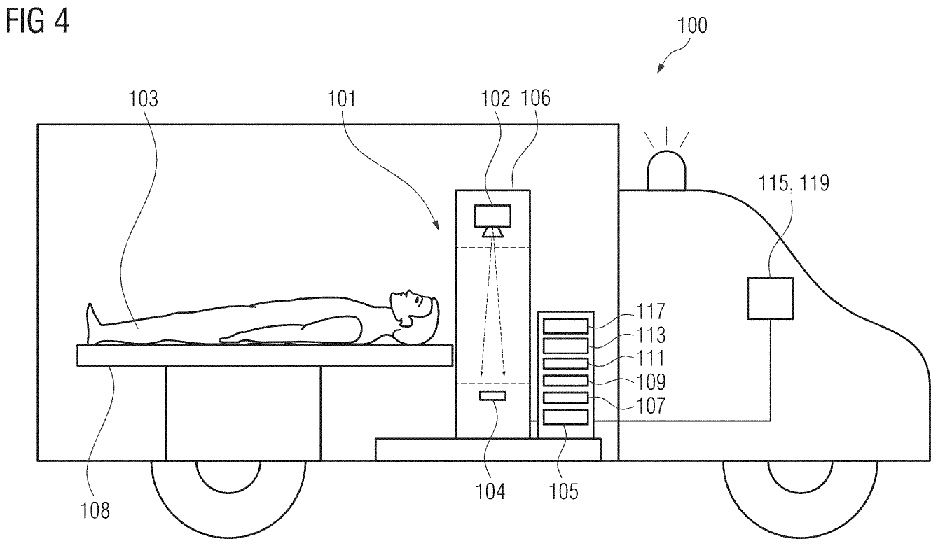

FIG. 4 shows a vehicle, in particular an ambulance, comprising a medical imaging device 101. In this example, the medical imaging device 101 is a computer tomography device with a measurement data recording unit 102,104 comprising an X-ray detector 104 and an opposing X-ray source 102, these being arranged in a gantry 106 which allows rotation of the measurement data recording unit 102,104 about a shared axis and therefore the recording of measurement data, i.e. in particular X-ray projection measurement data of a patient 103 from different angular ranges. Based upon this measurement data, it is then possible to reconstruct a first or second three-dimensional image data set, e.g. by way of a reconstruction algorithm for filtered back projection, or corresponding slice image data sets.

The patient 103 in this case is supported on a patient support apparatus 108 of the medical imaging device 101. For the purpose of recording the measurement data, the measurement data recording unit 102,104 is positioned relative to the patient 103 such that an examination region 6,7 can be scanned by the measurement data recording unit 102,104. Positioning can be achieved by moving or positioning the patient support apparatus 108 and/or by moving or positioning the measurement data recording unit 102,104, i.e. essentially the gantry 106.

The medical imaging device 101, here in the form of the CT device, is designed in particular to create a first image data set of the patient 103, i.e. to record measurement data and reconstruct the image data set based upon the measurement data. The medical imaging device 101 can be designed in particular to create a CT angiography image data set. Most advantageously, a CT angiography image data set makes it possible to check the image data set for the presence of a vessel blockage, and to identify the location of the vessel blockage and/or determine the type of blood vessel affected by the vessel blockage. Most advantageously therefore, an angiography image data set allows the image data set to be evaluated during the course of treating a stroke patient. However, other image data sets such as e.g. native CT image data sets without contrast medium support or perfusion image data sets are also conceivable in the context of the method, as are other application cases.

The vehicle 100 also has a first interface 105, which is designed to make the created image data set available for subsequent processing via the computing unit 107.

The vehicle 100 further comprises a computing unit 107, which is designed to determine a position 4 of the vehicle. The computing unit is also designed to determine a number of optional destination locations 1,2,3 for treatment of the patient 103 at least based upon the determined position 4 of the vehicle 100. The computing unit 107 is also designed for the third determination S5 of the preferred destination location from the number of optional destination locations 1,2,3 at least based upon the evaluated image data set and the determined position 4 of the vehicle 101.

The computing unit 107 can be realized in the form of a computer, a microcontroller or an integrated circuit. The computing unit 107 can have hardware elements or software elements, e.g. a microprocessor or a so-called FPGA (Field Programmable Gate Array). The computing unit 107 can also be realized as a plurality of interworking computers. The vehicle 100 also comprises a second interface 109, which is designed to output the preferred destination location that has been determined.

The vehicle 100 in this example also has a navigation system 115, which is designed to calculate navigation information relating to a navigation destination and to output the navigation information via an output unit 119, e.g. in the form of a display or a loudspeaker. The preferred destination location determined via the computing unit 107 can then be output to the navigation system 115 via the second interface 109 in particular. The navigation system 115 can optionally also comprise the position determining unit and make this available to the computing unit 107 in a retrievable manner.

The first interface 105 and the second interface 109 can be hardware or software interfaces (e.g. PCI bus, USB or Firewire).

The apparatus can also comprise at least one storage unit 117. A storage unit can also be part of the computing unit. This can be realized as non-permanent main memory (random access memory: RAM) or as permanent mass memory (hard disk, USB stick, SD card, solid-state disk). The at least one storage unit 117 can be used to buffer the first image data set for the evaluation step. The at least one storage unit 117 can also be used to hold e.g. a database of treatment locations and optionally information associated therewith for retrieval via the computing unit. The at least one storage unit 117 can also be used to buffer the determined number of optional destination locations 1,2,3 for a subsequent third determination step.

The vehicle 100 in this example also has a communication unit 111. For example, the communication unit 111 can allow data, i.e. information about the treatment locations or optional destination locations 1,2,3, to be requested via a radio network from a central unit, e.g. in respect of medical equipment or availability. The communication unit can be e.g. a telemedical system which allows the connection and data transfer via at least one radio network, in particular a mobile radio network.

The vehicle 100 in this example also has a position determining unit 113, e.g. in the form of a GPS receiver, which is designed to determine the position of the vehicle and make this available for retrieval via the computing unit for subsequent processing.

The subject matter of the invention is not limited to the example embodiments described above. Rather, further embodiment variants can be derived from the foregoing description by a person skilled in the art. In particular, the individual features of the invention and their different configurations as described with reference to the various example embodiments can also be combined together in other ways.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining a preferred destination location for a vehicle including a medical imaging device, comprising:
providing, via a first interface, an image data set relating to a patient, the image data set being created via the medical imaging device;
evaluating the image data set provided via a computing unit;
firstly determining a position of the vehicle via the computing unit;
secondly determining, via the computing unit, a number of optional destination locations for treatment of the patient, at least based upon the position of the vehicle determined;
thirdly determining, via the computing unit, the preferred destination location from the number of optional destination locations determined, at least based upon the image data set evaluated and the position of the vehicle determined; and
outputting the preferred destination location determined via a second interface.

2. The method of claim 1, wherein at least one parameter is determined during the secondly determining, the at least one parameter including at least one of:
medical equipment at at least one respective optional destination location, of the number of optional destination locations, for treating the patient,
availability of the medical equipment at at least one respective optional destination location for treating the patient,
travel route to at least one respective optional destination location, and
travel time to at least one respective optional destination location.

3. The method of claim 1, wherein the evaluating of the image data set includes checking the image data set for a presence of a vessel blockage of a blood vessel of the patient.

4. The method of claim 3, wherein, upon the checking indicating a presence of a vessel blockage, a location of the vessel blockage or a type of the blood vessel affected by the vessel blockage is determined.

5. The method of claim 1, wherein the evaluating includes application of a trained function.

6. The method of claim 1, wherein the image data set created is a CT angiography image data set.

7. The method of claim 1, wherein the firstly determining and the secondly determining are repeated, such that an updated number of optional destination locations is determined upon a position of the vehicle being updated.

8. The method of claim 1, wherein at least one of the firstly determining and the secondly determining is performed during the evaluating.

9. The method of claim 1, wherein navigation information for reaching the preferred destination location is calculated based upon the preferred destination location determined, and is output via an output unit.

10. A vehicle, comprising:
a medical imaging device, designed to create an image data set of a patient;
a first interface, designed to make the image data set created, available for subsequent processing;
at least one processor, designed to
evaluate the image data set created,
determine a position of the vehicle,
determine a number of optional destination locations for treatment of the patient, at least based upon the position of the vehicle determined,
select a preferred destination location from the number of optional destination locations, at least based upon the image data set evaluated and the position of the vehicle determined; and
a second interface, designed to output the preferred destination location selected.

11. The vehicle of claim 10, wherein at least one parameter is determined by the at least one processor in determining of the number of optimal destination locations, the at least one parameter including at least one of:
medical equipment at at least one respective optional destination location, of the number of optional destination locations, for treating the patient,
availability of the medical equipment at at least one respective optional destination location for treating the patient,
travel route to at least one respective optional destination location, and
travel time to at least one respective optional destination location.

12. A storage unit of a computing unit of a vehicle, storing a computer program including program sections for executing the method of claim 1 when the program sections are executed by the computing unit.

13. A non-transitory computer program product storing a computer program, directly loadable into a storage unit of a computing unit of a vehicle, including program sections for executing the method of claim 1 when the program sections are executed by the computing unit.

14. A non-transitory computer-readable storage medium storing program sections, readable and executable by a computing unit to execute the method of claim 1 when the program sections are executed by the computing unit.

15. The method of claim 2, wherein the evaluating of the image data set includes checking the image data set for a presence of a vessel blockage of a blood vessel of the patient.

16. The method of claim 15, wherein, upon the checking indicating a presence of a vessel blockage, a location of the vessel blockage or a type of the blood vessel affected by the vessel blockage is determined.

17. The method of claim 2, wherein the evaluating includes application of a trained function.

18. The method of claim 3, wherein the evaluating includes application of a trained function.

19. The method of claim 2, wherein navigation information for reaching the preferred destination location is calculated based upon the preferred destination location determined, and is output via an output unit.

20. The vehicle of claim 10, wherein the at least one processor is designed to evaluate the image data set by checking the image data set for a presence of a vessel blockage of a blood vessel of the patient.
